(19) United States
(12) Patent Application Publication
(10) Pub. No.: US 2026/0034258 A1
(43) Pub. Date: Feb. 5, 2026

(54) APPARATUS, PLANT AND METHOD FOR THE INDUSTRIAL WASHING OF SURGICAL INSTRUMENTS

(71) Applicant: STEELCO S.P.A., RIESE PIO X (TV) (IT)

(72) Inventors: Alessandro CAPRARA, ISOLA DELLA SCALA (VR) (IT); Paolo RIGHETTINI, DALMINE (BG) (IT)

(73) Assignee: STEELCO S.P.A., RIESE PIO X (TV) (IT)

(21) Appl. No.: 18/998,821

(22) PCT Filed: Jul. 25, 2023
(86) PCT No.: PCT/IT2023/050173

§ 371 Date: Jan. 27, 2025

(30) Foreign Application Priority Data
Jul. 27, 2022 (IT) 102022000014005

(51) Int. Cl. A61L 2/24; A61L 2/025; A61L 2/04

(52) U.S. Cl. CPC A61L 2/24; A61L 2/025; A61L 2/04; A61L 2202/17; A61L 2202/24

(57) ABSTRACT

Washing apparatus (10) for washing surgical instruments (11) disposed in one or more containers (12), comprising movement means (16) which define at least one first advance line (L1) having an inlet end (E) and an outlet end (U) and are configured to make the containers (12) advance along the first advance line (L1), a plurality of first washing stations (25) disposed along the first advance line (L1), and first pick-up means (70) to pick up at least one container (12) from the first advance line (L1) and position it in a washing station (25).

### FIELD OF THE INVENTION

The present invention concerns an apparatus, a plant and a method for the industrial washing of surgical instruments, for example coming from one or more operating theaters.

### BACKGROUND OF THE INVENTION

The instruments used during surgical operations are normally supplied to the medical team in kits, or sets, each comprising a determinate number and type of surgical instruments. For example, each kit contains instruments dedicated to a specific surgical procedure.

Normally, to avoid contamination during the washing and sterilization steps, a specific kit must never be dismantled, so that at the end of the wash a kit always contains the same identical instruments contained at the end of the surgical operation.

Usually, surgical instruments are washed in thermal disinfection washing machines, or instrument washers, and they are sterilized in steam sterilizers, for example autoclaves.

In addition, before the actual washing of the surgical instruments in the instrument washers, they are subjected to a preliminary washing with the aim of removing any solid residues of blood, or other material deriving from the surgical operation, deposited on the surgical instruments to be washed.

The preliminary wash provides to subject the instruments to a cold wash, to an ultrasonic wash and possibly also to a manual wash using manual apparatuses such as for example brushes to remove coagulated blood or other material. In fact, usually, not all the instruments coming from an operating theater have been used and need a thorough preliminary wash.

In the preliminary wash, an operator visually selects the surgical instruments and chooses whether to wash each of them in a cold washing machine, or to wash them manually.

This solution, although widely used, is not very efficient and productive, especially when it is required to wash a large number of instruments, for example in hospitals or surgeries which perform many surgical operations per day.

Document US 2021/002013 A1 describes a method and a system for reconditioning a medical instrument.

Document US 2021/236676 A1 describes an apparatus for treating medical instruments is described.

Document EP 3 009 201 A1 describes a washing apparatus, in particular a thermal disinfection apparatus.

There is therefore a need to perfect an apparatus, a plant and a method for the industrial washing of surgical instruments which can overcome at least one of the disadvantages of the state of the art.

To do this it is necessary to solve the technical problem of being able to automate the passage of instruments from one washing machine to another, at the same time guaranteeing a high level of cleanliness.

In particular, one purpose of the present invention is to provide an apparatus, a plant and a method for the industrial washing of surgical instruments which can reduce the time required for washing the instruments.

Another purpose of the present invention is to provide an apparatus, a plant and a method for the industrial washing of surgical instruments which does not require the presence of specialized personnel.

Another purpose of the present invention is to provide an apparatus, a plant and a method for the industrial washing of surgical instruments which is reliable and easy to use.

Another purpose of the present invention is to provide an apparatus, a plant and a method for the industrial washing of surgical instruments which allows to wash surgical instruments without dismantling the kits to which they belong, and to trace the instruments which make up the kits.

Another purpose of the present invention is to provide an apparatus, a plant and a method for the industrial washing of surgical instruments which is extremely rapid, efficient and in which it is possible to increase productivity and possibly to differentiate the washes performed, providing in particular at least two lines for making the objects advance along the apparatus, and hence two washing lines.

The Applicant has devised, tested and embodied the present invention to overcome the shortcomings of the state of the art and to obtain these and other purposes and advantages.

### SUMMARY OF THE INVENTION

The present invention is set forth and characterized in the independent claims. The dependent claims describe other characteristics of the present invention or variants to the main inventive idea.

In accordance with the above purposes and to resolve the technical problem disclosed above in a new and original way, also achieving considerable advantages compared to the state of the prior art, a washing apparatus for washing surgical instruments disposed in one or more containers, according to the present invention comprises movement means which are configured to make the containers advance automatically along at least one first advance line, a plurality of first washing stations associated with the at least one first advance line, each of which is configured to perform a washing operation on the surgical instruments, and first pick-up means configured to automatically pick up at least one of the containers from the at least one first advance line and position it in one of the first washing stations, and vice versa.

In accordance with a possible aspect described here, the movement means can be commanded by a central control unit and configured to make the containers advance automatically also along a second advance line which is autonomous with respect to the first advance line. Moreover, the apparatus also comprises one or more second washing stations associated with the second advance line, and second pick-up means configured to automatically pick up at least one of the containers from the at least one second advance line and position it in one of the second washing stations, and vice versa.

In accordance with another aspect of the present invention, the washing stations comprise at least one of either a cold washing station, an ultrasonic washing station and a targeted washing station.

In accordance with another aspect of the present invention, the at least one advance line comprises an inlet end and an outlet end, and the apparatus also comprises an inlet station disposed in correspondence with the inlet end and configured to allow to position the containers on the movement means, and an outlet station disposed in correspondence with the outlet end and configured to allow to pick up the containers from the movement means.

According to another aspect of the invention, the inlet station comprises a robotized anthropomorphic arm configured to pick up a container from a collection zone and position it in the first advance line or in the second advance line.

In accordance with another aspect of the present invention, the outlet station comprises grouping means configured to position the containers present in the outlet station on at least one mobile support member configured to be inserted in a machine for thermal disinfection, or an instrument-washing machine.

The grouping means can comprise two lifting devices, each of which is associated with a movement member of the movement means and configured to receive a flow of containers coming from the respective movement member.

The grouping means can comprise a robotized anthropomorphic arm configured to pick up the containers from the outlet station and position them in a grouping zone.

In accordance with another aspect of the present invention, the apparatus also comprises a plurality of closing walls containing inside them at least the movement means, the washing stations and the pick-up means, and having an inlet aperture in correspondence with the inlet end and an outlet aperture in correspondence with the outlet end.

With each container there can be associated a respective recognition element encoding the information on the instruments contained therein and it can comprise recognition means configured to recognize the recognition element of each container the instant in which the latter is positioned on the movement means.

The pick-up means comprise a plurality of pick-up members, each of which is interposed between an advance line and a washing station, wherein each pick-up member comprises a frame on which a mobile gripping member is mounted, commanded by the central control unit and configured to displace a container from an advance line toward the associated washing station, and vice versa.

The gripping member can be substantially C-shaped and its internal portion mirrors, negatively, at least one portion of the external perimeter of a container.

The frame can comprise a temporary storage zone on which a container can be disposed so as to compensate for any fluctuations in the flow of containers introduced into the washing apparatus.

The present invention also concerns a plant for washing surgical instruments comprising an apparatus according to the present invention, at least one machine for the thermal disinfection of the surgical instruments and additional movement means configured to automatically move the surgical instruments coming out of the apparatus toward the at least one thermal disinfection machine.

The present invention also concerns a washing method for washing surgical instruments which are disposed in one or more containers, which comprises:
an introduction step in which the containers are positioned on the first and second advance line;
a feed step in which first containers are made to advance automatically on the first advance line and, simultaneously, second containers are made to advance on the second advance line;
a first pick-up step in which at least one container is automatically picked up from the first advance line and positioned in one of the first washing stations and at least one container is automatically picked up from the second advance line and positioned in one of the second washing stations;
a washing step in which a washing of the surgical instruments contained in the containers is performed.

The first containers can be suitable to contain a first type of the surgical instruments and the second containers can be suitable to contain a second type of the surgical instruments. In this case, therefore, thanks to the present method it is possible to perform the washing of different instruments, even simultaneously, a first type on the first line and a second type on the second line.

In accordance with another aspect of the present invention, the at least one washing operation is selected from a cold washing operation, an ultrasonic washing operation and a targeted washing operation.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a schematic top view of an apparatus in accordance with an embodiment of the present invention;

FIGS. 2 and 4 are schematic top views of respective parts of the apparatus of FIG. 1;

FIGS. 3, 5 and 6 are schematic three-dimensional views of respective parts of the apparatus of FIG. 1;

FIG. 7 is a schematic top view of a plant in accordance with an embodiment of the present invention;

FIG. 8 is a schematic representation of an electrical diagram of an apparatus according to an embodiment of the present invention.

We must clarify that in the present description the phraseology and terminology used, as well as the figures in the attached drawings also as described, have the sole function of better illustrating and explaining the present invention, their function being to provide a non-limiting example of the invention itself, since the scope of protection is defined by the claims.

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

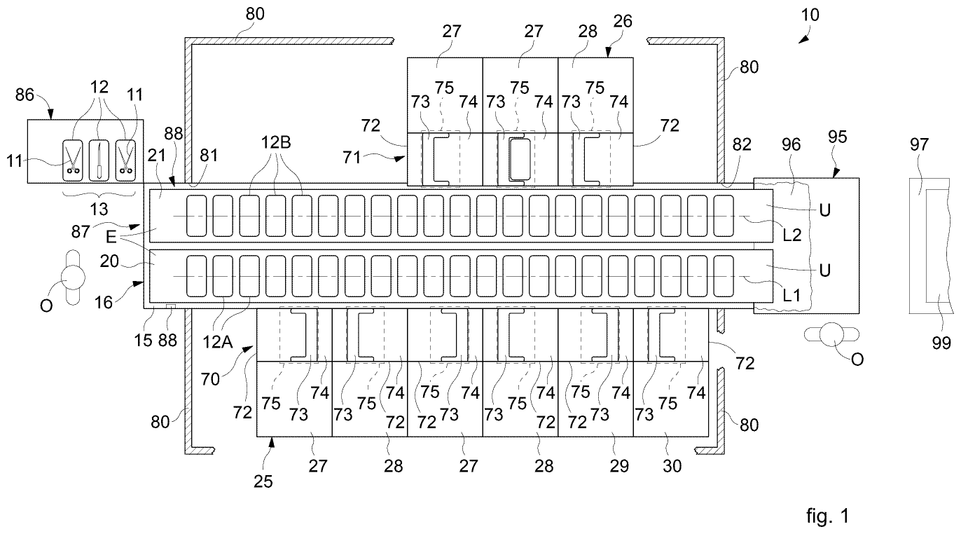

With reference to FIG. 1, an apparatus 10 according to the present invention is configured to automatically perform a wash, for example a preliminary wash, of surgical instruments 11, hereafter instruments 11.

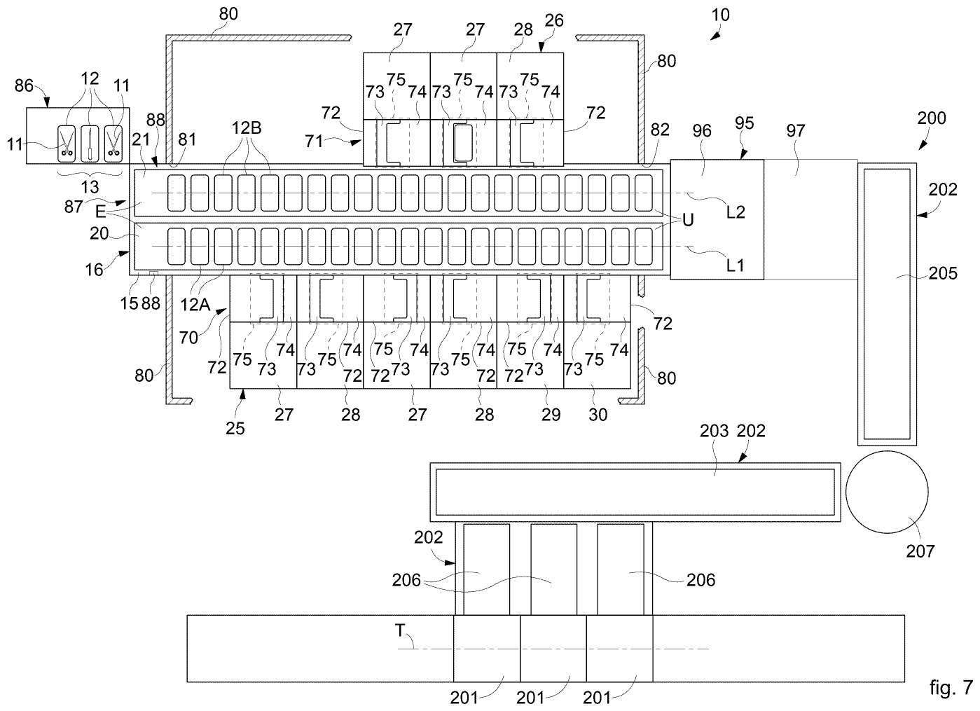

Preferably, the apparatus 10 of the present invention is inserted in a washing plant 200 (FIG. 7), upstream of a thermal disinfection machine 201 configured to treat the instruments 11 washed by means of the apparatus 10 of the present invention.

The instruments 11 to be washed come from one or more operating theatres and are grouped into kits 13, or sets, of surgical instruments 11. In particular, here and hereafter in the description, with the term “kit” 13 we mean a certain group of surgical instruments 11.

The apparatus 10 also comprises a plurality of containers 12 and the instruments 11 of each kit 13 are divided into one or more containers 12, which are open at the top and provided with holes to allow the instruments 11 contained therein to be washed.

For example, a container 12 can be made of metal and its walls can be conformed as a mesh, or a net, in order to define a plurality of passage apertures for water or other washing fluids.

With each container 12 there is associated a respective recognition element 14 encoding the information on the instruments 11 contained therein. This recognition element 14 also encodes the kit 13 to which these instruments 11 belong.

By way of example only, the recognition element 14 may be of the barcode, QR code, RFID, Bluetooth or other type configured to transmit data through electromagnetic waves, or similar and comparable types.

With reference to FIG. 1, the apparatus 10 comprises a support structure 15 for movement means 16 which define a first advance line L1. The movement means 16 can also define a second advance line L2.

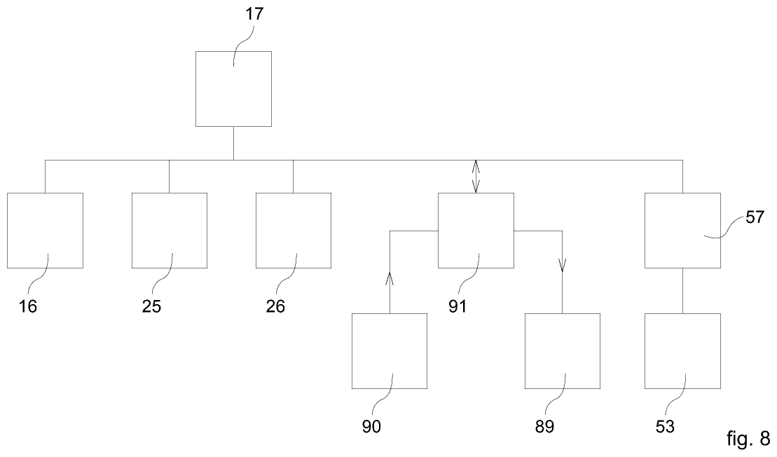

The movement means 16 are commanded by a central control unit 17 (FIG. 8). The movement means 16 comprise a first movement member 20 (FIG. 1) defining the first advance line L1 and configured to make first containers 12A advance along it. Moreover, the movement means 16 comprise a second movement member 21 (FIG. 1) defining the second advance line L2 and configured to make second containers 12B advance along it.

The central control unit 17 (FIG. 8) is configured to make each container 12 advance, by a certain step and with a certain frequency, along the respective advance line L1, L2, therefore in particular to move the movement members 20, 21 (FIG. 1).

The advance lines L1, L2 have an inlet end E from which the containers 12 containing the instruments 11 to be washed are introduced, and an outlet end U from which the containers 12 containing the washed instruments 11 are picked up

In the embodiments shown in the attached drawings, the inlet end E and the outlet end U correspond for both the advance lines L1, L2. However, in other possible embodiments, the ends of one advance line may not correspond to the ends of another advance line. Moreover, although in the examples given the advance lines L1, L2 have a rectilinear shape, in other embodiments they may have another shape, for example curvilinear or defined by any combination of rectilinear and/or curved segments whatsoever.

Each movement member 20, 21 comprises at least one respective conveyor belt moved by an electric motor (not shown). In some embodiments, each movement member 20, 21 comprises at least a plurality of conveyor belts disposed in series and moved by respective independent electric motors (not shown). The person of skill in the art will understand, however, that in other possible embodiments the movement members 20, 21 can comprise roller members, rail members, rack members, chain members, belt members, or other similar or comparable members.

The apparatus 10 comprises a plurality of washing stations 25, 26 each of which is controlled by the central control unit 17 and configured to perform a washing operation on the instruments 11 contained in one or more containers 12.

In particular, first washing stations 25 are disposed side by side to the first movement member 20 along the first advance line L1 and second washing stations 26 are disposed side by side to the second movement member 21 along the second advance line L2.

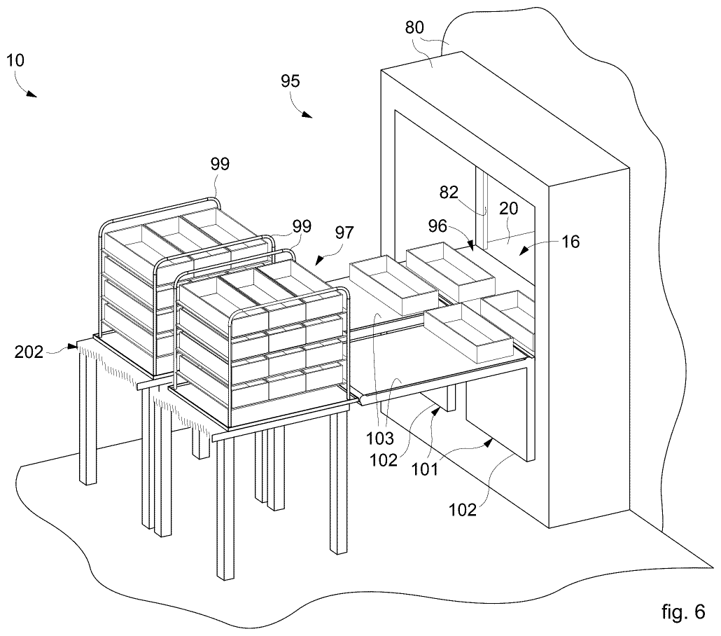

In the example given here, the washing stations 25, 26 can be cold washing stations 27 (FIG. 3), ultrasonic washing stations 28 and targeted washing stations 29, 30 (FIG. 5), which will be described below.

A cold washing station 27 (FIG. 3) comprises an external structure 31 having a front aperture 32 and containing a cold washing machine 33, of a type known per se and which will not be described in detail.

The cold washing station 27 also comprises a support member 35 mobile between the inside of the cold washing machine 33 and the front aperture 32. The support member 35 comprises a plurality of support portions 36 for the containers 12 containing the instruments 11 to be washed.

An ultrasonic washing station 28 comprises an external structure 41 having a front aperture 42 and containing an ultrasonic washing machine 43, of a type known per se and which will not be described in detail.

In addition, the ultrasonic washing station 28 also comprises a support member 45 mobile between the inside of the ultrasonic washing machine 43 and the front aperture 42 and comprising a plurality of support portions 46 for positioning the containers 12 containing the instruments 11 to be washed.

A targeted washing station 29, 30 (FIG. 5) is configured to perform a targeted wash of the instruments 11 contained in a container 12. Here and hereafter, by “targeted wash” we mean a wash carried out by means of a jet of washing fluid, for example water, or water and bicarbonate, or steam, or hydrogen peroxide, directed directly onto the dirt agglomerates present on each instrument 11 present in a container 12.

In order to be able to perform a complete targeted wash on both sides of the instruments 11, the apparatus 10 comprises both an upper targeted washing station 29 and also a lower targeted washing station 30.

Each targeted washing station 29, 30 comprises an external structure 51 having a front aperture 52 and a rest plane 54 for a container 12. In each targeted washing station 29, 30 there is also disposed a viewing device 53 and a device 55 for delivering the pressurized liquid mounted on a movement device 56.

In the example given here, the movement device 56 is a Cartesian robot and it is capable of moving the delivery device along three reciprocally independent axes.

The movement device 56 is commanded by a processing unit 57 configured to receive and process the images taken by the viewing device 53 in order to recognize the dirt residues on the instruments 11 which are disposed in the container 12 lying on the rest plane 54. In addition, the processing unit 57 is also configured to determine the position of the recognized dirt residues and command the movement device 56 to position the delivery device 55 in such a way that the jet delivered by the latter hits the dirt residues on the instruments 11.

In the upper targeted washing station 29 the viewing device 53, the delivery device 55 and the movement device 56 are disposed above the rest plane 54.

Instead, in the lower targeted washing station 30 the viewing device 53, the delivery device 55 and the movement device 56 are disposed below the rest plane

Preferably, the lower washing station 30 also comprises a closing member 58 configured to close the upper aperture of the container 12 that is disposed inside the lower washing station 30.

In particular, the closing member 58 has the function of preventing the escape of the instruments 11 contained in the container 12 that is disposed inside the lower targeted washing station 30 when they are hit by a jet of water coming from the bottom upward.

The closing member 58 comprises an arm 59 mobile at least in the vertical direction, to which there is attached a closing plate 60 suitable to overlap with the upper aperture of each container 12.

The combination of the washing stations 25, 26 along an advance line L1, L2 defines the level of cleanliness that can be obtained for the instruments 11 that travel along the advance line L1, L2 considered.

In this way, it is possible to differentiate the type of wash and the level of cleanliness that can be achieved along the first or second advance line, respectively L1 and L2.

By way of example only, in the embodiment of FIG. 1, a cold washing station 27, an ultrasonic washing station 29, another cold washing station 27, another ultrasonic washing station 29, an upper targeted washing station 29 and a lower washing station 30 are disposed in series along the first advance line L1. Instead, two cold washing stations 27 and possibly one or more ultrasonic washing stations 28 are disposed along the second advance line L2.

In this case, first containers 12A are fed along the first advance line L1, containing instruments 11 that need to be thoroughly washed, for example instruments 11 soiled with blood or other material deriving from operations on a human body. Instead, second containers 12B are fed along the second advance line L2, containing instruments 11 that require only a surface wash, for example instruments that were not used in the operating room but that were present inside it.

The person of skill in the art will easily understand that the combination of the washing stations 25, 26 along the advance lines L1, L2 can also be very different from the embodiments described here, without thereby departing from the scope of the present invention. In fact, the combination of the washing stations 25, 26 along the advance lines L1, L2 may depend on the desired productivity and also on the washing procedure required from the apparatus 10.

The apparatus 10 also comprises pick-up means 70, 71 (FIGS. 1 and 3) configured to automatically pick up the containers 12 from the advance lines L1, L2 and to position them in the washing stations 25, 26.

In particular, the apparatus 10 comprises first pick-up means 70 configured to automatically pick up the containers 12 from the first advance line L1 and position them in the first washing stations 25, and second pick-up means 71 configured to automatically pick up the containers 12 from the second advance line L2 and position them in the second washing stations 26.

The pick-up means 70, 71 comprise a plurality of pick-up members 72, each of which is interposed between an advance line L1, L2 and a washing station 25, 26.

Each pick-up member 72 comprises a frame 74 on which a mobile gripping member 73 is mounted, moved by a drive member (not shown) commanded by the central control unit 17, and configured to displace a container 12 from an advance line L1, L2 toward the associated washing station 25, 26 and vice versa.

In this specific case, the gripping member 73 is substantially C-shaped and its internal portion mirrors, negatively, at least one portion of the external perimeter of a container 12. The gripping member 73 is also moved in a direction that is orthogonal to the advance line L1, L2 with which it is associated.

Preferably, each frame 74 also comprises a temporary storage zone 75 on which a container 12 can be disposed so as to compensate for any fluctuations in the flow of containers 12 introduced into the apparatus 10.

In the embodiments shown here, the storage portion 75 is defined by a cavity created on the upper portion of the frame 74.

In addition, the pick-up means 70, 71 can also comprise transfer members 77 (FIG. 3) configured to transfer one or more containers 12 from one washing station 25, 26 to another adjacent washing station 25, 26.

Each transfer member 77 comprises a gripping member 79 moved by a drive member (not shown) commanded by the central control unit 17 and configured to displace a container 12 from one washing station 25, 26 to another adjacent one.

In this specific case, the gripping member 79 is moved by a Cartesian robot 78 interposed between two adjacent washing stations 25, 26

For example, in the embodiment of FIG. 1, between a cold washing station 27 and an adjacent ultrasonic washing station 28 there can be disposed a transfer member 77 so that the passage of a container 12 from the cold washing station 27 to the ultrasonic washing station 28 can occur without the container 12 returning to the advance line L1, L2 on which it was travelling.

The apparatus 10 also comprises a plurality of walls 80 (FIG. 1) containing inside them the movement means 16, the washing stations 25, 26 and the pick-up means 70, 71

The walls 80 allow to separate the inside of the apparatus 10 from the environment in which it is positioned, in order to guarantee the conditions of hygiene and cleanliness inside it.

The walls 80 also comprise an inlet aperture 81 in correspondence with the inlet end E of the advance lines L1, L2 and an outlet aperture 82 in correspondence with the outlet end U of the advance lines L1, L2.

The apertures 81, 82 of the walls 80 are of a shape and size suitable to allow the introduction and extraction of the containers 12 therefrom.

The apparatus 10 also comprises an inlet station 85 disposed in proximity to the inlet end E and configured to allow the positioning of each container 12 on the movement means 16.

The inlet station 85 comprises both a collection zone 86 in which the containers 12 are disposed, and also a feeding zone disposed in correspondence with the inlet aperture 81.

The apparatus 10 also comprises recognition means 88, located in particular upstream of, or in correspondence with, the inlet aperture 81, which are configured to recognize the recognition element 14 of each container 12 the instant in which the latter is positioned on the movement means 16

The recognition means 88 are also configured to transmit to the central control unit 17 information relating to the instant in which the container 12 is positioned on the movement means 16, in order to allow to determine the next position of the container 12 considered.

In this way, the central control unit 17 can process the information on the instant in which a container 12 is positioned on the movement means 16 and the extent of the movement commanded to each movement member 20, 21 so as to be able to determine the position of each container 12 present on the advance lines L1, L2.

The transfer of the containers 12 from the collection zone 86 to the feeding zone 87 can be performed manually by a user O, who decides whether to position each container 12 in the first advance line L1 or in the second advance line L2 depending on the level of dirtiness of the instruments 11 present in the container 12 under consideration and the combinations of washing stations 25, 26 associated with each advance line L1, L2.

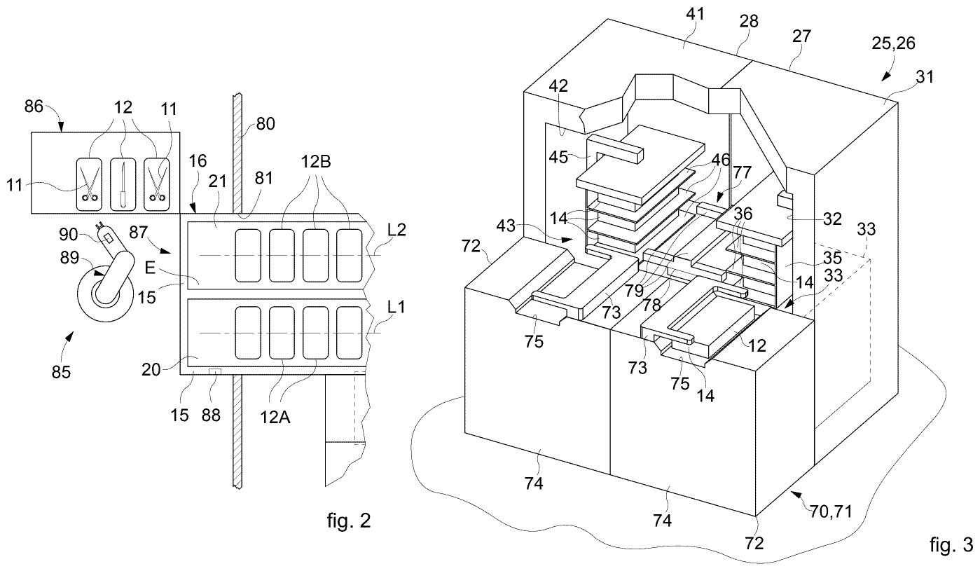

Alternatively, in another embodiment schematically shown in FIG. 2, the inlet station 85 comprises a robotized anthropomorphic arm 89 configured to pick up a container 12 from the collection zone 86 and position it in the first advance line L1 or in the second advance line L2.

In this case, a viewing device 90 configured to capture images relating to the container 12 manipulated by the robotized anthropomorphic arm 89 can be associated with the robotized anthropomorphic arm 89.

Moreover, the viewing device 90 is connected to a processing unit 91, communicating also with the central control unit 17, and configured to process the images taken by the viewing device 90 and command the robotized anthropomorphic arm 89. The processing unit 91 is configured to detect whether there are dirt residues on the instruments 11 disposed inside the container 12 manipulated by the robotized anthropomorphic arm 29, in order to decide on which advance line L1, L2 to dispose the container 12.

The apparatus 10 also comprises an outlet station 95 (FIG. 1) disposed in correspondence with the outlet aperture 82 and configured to allow to pick up each container 12 from the advance lines L1, L2, that is, from the movement members 20, 21.

The outlet station 95 comprises an extraction zone 96, disposed in correspondence with the outlet end U of the advance lines L1, L2, in which the containers 12 coming from the movement members 20, 21 are left.

Optionally, the outlet station 95 also comprises a grouping zone 97 configured to allow to dispose the containers 12 present in the extraction zone 96 on a mobile support member 99, for example a trolley having a plurality of rest planes, which is configured to be inserted in a machine 201 for the thermal disinfection of the instruments 11, the machine 201 being disposed downstream of the apparatus 10.

The transfer of the containers 12 from the extraction zone 96 to the grouping zone 97 occurs manually and is performed by a user who positions each container 12 in a mobile support member 99.

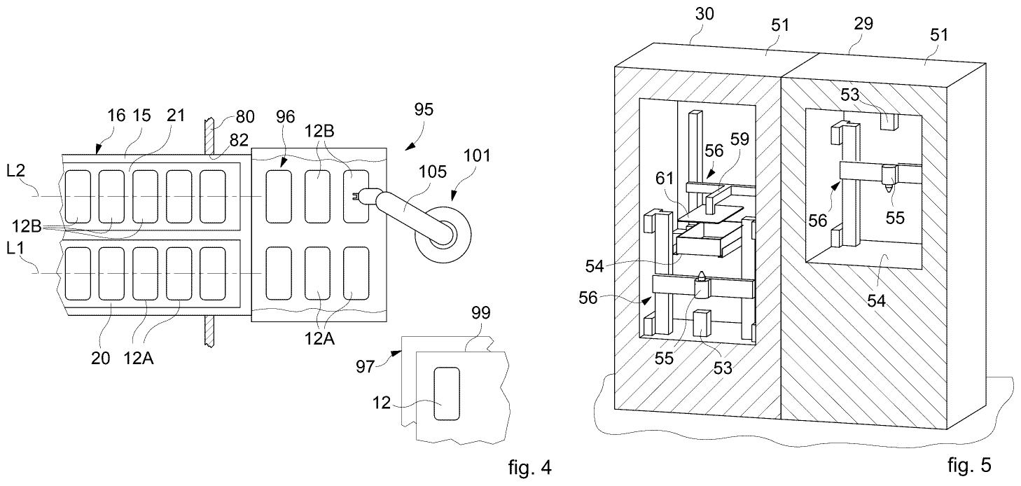

Alternatively, in other embodiments schematically shown in FIGS. 4 and 6, the outlet station 95 comprises grouping means 101 configured to position each container 12 present in the extraction zone 96 on the mobile support member 99 disposed in the grouping zone 97.

In the example shown in FIG. 6, the grouping means 101 comprise two lifting devices 102, each of which is associated with a movement member 20, 21 and configured to receive a flow of containers 12 coming from the respective movement member 20, 21.

Each lifting device 102 comprises a rest plane 103 on which the containers 12 coming from the respective movement member 20, 21 lie, and lifting members (not shown) configured to align the rest plane 103 to a support portion of the mobile support member 99.

Preferably, the rest plane 103 consists of the upper part of a conveyor belt suitable to move the containers 12 from the extraction zone 96 toward the mobile support member 99.

In another example shown in FIG. 4, the grouping means 101 comprise a robotized anthropomorphic arm 105 configured to pick up the containers 12 from the outlet station 95, which are located in particular in the extraction zone 96, and position them in the grouping zone 97 of the outlet station 95, in particular on the mobile support member 99.

The present invention also concerns a plant 200 (FIG. 7) for washing surgical instruments 11, which comprises an apparatus 10 as described above and at least one thermal disinfection machine 201 disposed downstream of the apparatus 10.

Preferably, the plant 200 comprises a plurality of thermal disinfection machines 201 aligned along an alignment axis T, and transport means 202 defining a first transport segment 203 parallel to the alignment axis T and a second transport segment 205 running from the outlet station 95 of the apparatus 10 of the present invention to the beginning of the first transport segment 203.

Optionally, in front of each thermal disinfection machine 201 there is disposed another insertion segment 206 of the transport means 202 which is configured to insert in the respective thermal disinfection machine 201 a mobile support member 99 travelling on the first transport segment 103.

In addition, between the first 103 and the second transport segment 105 there is disposed a platform 207 configured to allow the mobile supports 99 that travel on the transport means 202 to change direction.

The operation of the apparatus 10 according to the present invention, which corresponds to the method according to the present invention, comprises the following steps.

The method provides an introduction step in which the containers 12 containing the instruments 11 to be washed are positioned on the movement means 16.

This introduction step can be performed manually by an operator who positions each container 12 in the first advance line L1 or in the second advance line L2, depending on the state of cleanliness of the surgical instruments 11 present in the container 12 and the combinations of washing stations 25, 26 associated with each advance line L1, L2.

Alternatively, in the embodiments which provide it, this introduction step can be performed automatically by a robotized anthropomorphic arm 89.

Optionally, when a container 12 is positioned on the movement means 16, a recognition step also occurs, in which the recognition means 88 recognize the recognition element 14 associated with that determinate container 12 and transmit an electrical signal to the control unit 17 comprising the information contained in the recognition element 14 and temporal information relating to the instant in which the container 12 was positioned on the movement means 16.

Subsequently, the method provides a feeding step in which the container 12 is made to advance along the advance line L1, L2 on which it has been positioned.

For example, in the embodiment schematically shown in FIG. 1, a first container 12A, containing at least one dirty surgical instrument 11, is made to advance along the first advance line L1 while a second container 12B, containing clean surgical instruments 11, is made to advance along the second advance line L2.

The advance of the containers along the advance lines L1, L2 is commanded by the central control unit 17, which in turn commands the movement members 20, 21.

For example, the advance can be stepwise or continuous.

The method also provides a first pick-up step for each container 12 which is automatically picked up from the advance line L1, L2 along which it advances and positioned in a washing station 25, 26.

By way of example, when a first container 12A advancing along the first advance line L1 is in front of a cold 27, or ultrasonic 28, or targeted 29, 30 washing station, the control unit 17 commands the respective pick-up member 72 to position that first container 12A in the station it is in front of.

On the other hand, again by way of example, when a second container 12B advancing along the second advance line L2 is in front of a cold 27 or ultrasonic 28 washing station, the control unit 17 commands the respective pick-up member 72 to position that second container 12B in the station it is in front of.

Optionally, each container 12 can stop in the temporary storage zone 75 before being disposed on the mobile support 35, 45 in order to absorb and compensate for any fluctuations in the flow of containers introduced into the apparatus 10.

When a washing station 27, 28, 29, 30 is sufficiently filled, the method provides a washing step in which a washing operation of the instruments 11 contained in the containers 12 which are disposed in the washing station 27, 28, 29, 30 is performed.

The washing step can provide a cold washing operation, an ultrasonic washing operation or a targeted washing operation, depending on the washing station considered.

The method also provides a second pick-up step in which a container 12 disposed inside a washing station 27, 28, 29, 30 is automatically extracted therefrom.

The second pick-up step can provide to pick up the container 12 from the washing station 27, 28, 29, 30 in which it is located in order to position it once again on the advance line L1, L2 along which it was advancing.

Alternatively, the second pick-up step can provide to pick up the container 12 from the washing station 27, 28, 29, 30 in which it is located in order to position it in another washing station 27, 28, 29, 30. In this case, the method can provide to perform an additional washing operation in the new washing station 27, 28, 29, 30 in which the container 12 is located. Then, the method provides a new pick-up operation to pick up the container 12 from the washing station 27, 28, 29, 30 in which it is located and position it once again on the advance line L1, L2 along which it was advancing.

When the container 12 has been once again positioned on the advance line L1, L2 along which it was advancing, the method provides to make the container 12 advance along the advance line L1, L2 toward a new washing station 27, 28, 29, 30 or toward the outlet station 95.

Below, purely by way of example, we will refer to the embodiment shown in FIG. 1 and describe the path of a first container 12A positioned on the first advance line L1 which is made to advance until the first cold washing station 27.

Subsequently, the first container 12A is picked up from the first advance line L1 and is inserted inside the cold washing station 27 in which it is subjected to a cold washing operation.

At the end of the cold washing operation, the first container 12A is extracted from the cold washing station 27 and inserted into the ultrasonic washing station 28 adjacent to the cold washing station 27 by means of the transfer means 77 interposed between them.

The first container 12A is then subjected to an ultrasonic washing operation and, once this has ended, it is extracted from the ultrasonic washing station 28 and once again taken onto the first advance line L1.

We must clarify that these washing operations can be performed in the same way on other first containers 12A fed along the first advance line 12, also in the other two cold 27 and ultrasonic 28 washing stations present along it.

When the first container 12A is once again on the first advance line L1, it is made to advance until it is in front of the upper targeted washing station 29. At this point, the first container 12A is picked up from the first advance line L1 and is inserted inside the upper targeted washing station 29 in which it undergoes a targeted washing operation with a jet of water coming from the top down.

At the end of the targeted washing operation, the first container 12A is extracted from the upper targeted washing station 29 and it is once again taken onto the first advance line L1.

When the first container 12A is once again on the first advance line L1, it is made to advance until it is in front of the lower targeted washing station 30. At this point, the first container 12A is picked up from the first advance line L1 and is inserted inside the lower targeted washing station 30 in which it undergoes a targeted washing operation with a jet of water coming from the bottom upward.

At the end of this last targeted washing operation, the first container 12A is picked up from the lower targeted washing station 30 and it is once again taken onto the first advance line L1.

At this point, the first container 12 is made to advance along the first advance line L1 and is taken toward the outlet station 95 from which it is extracted, manually or automatically, and positioned on the mobile support member 99.

Preferably, the mobile support member 99 will subsequently be used to take a plurality of containers 12 toward a thermal disinfection machine 201 disposed downstream of the apparatus 10, for example as in the plant 200 according to the present invention.

In the following, purely by way of example, we will refer to the embodiment shown in FIG. 1 and describe the path of a second container 12B positioned on the second advance line L2, which is made to advance until a cold washing station 27 positioned along the second advance line L2.

Then, the second container 12B is picked up from the second advance line L2 and is inserted inside the cold washing station 27 in which it is subjected to a cold washing operation.

Optionally, at the end of the cold washing operation, the second container 12B is extracted from the cold washing station 27 and inserted in the ultrasonic washing station 28 adjacent to the cold washing station 27, by means of the transfer means 77 interposed between them.

The second container 12B is then subjected to an ultrasonic washing operation and, once this has ended, it is extracted from the ultrasonic washing station 28 and once again taken onto the second advance line L2.

At this point, the second container 12 is made to advance along the first advance line L1 and is taken toward the outlet station 95 from which it is extracted, manually or automatically, and positioned on the mobile support member 99 together with other second containers 12B.

Preferably, the mobile support member 99 will subsequently be used to take a plurality of second containers 12B toward a thermal disinfection machine 201 disposed downstream of the apparatus 10.

It is clear that modifications and/or additions of parts may be made to the apparatus 10, to the plant 200 and to the method as described heretofore, without departing from the field and scope of the present invention, as defined by the claims.

It is also clear that, although the present invention has been described with reference to some specific examples, a person of skill in the art will be able to achieve other equivalent forms of apparatus 10, plant 200 and method for washing surgical instruments, having the characteristics as set forth in the claims and hence all coming within the field of protection defined thereby.

In the following claims, the sole purpose of the references in brackets is to facilitate their reading and they must not be considered as restrictive factors with regard to the field of protection defined by the very claims.

1. Washing apparatus (10), for washing surgical instruments (11) disposed in one or more containers (12), comprising movement means (16) which are configured to make said containers (12) advance automatically along at least one first advance line (L1), a plurality of first washing stations (25) associated with said at least one first advance line (L1), each of which is configured to perform a washing operation on said surgical instruments (11), and first pick-up means (70) configured to automatically pick up at least one of said containers (12) from said at least one first advance line (L1) and position it in one of said first washing stations (25), and vice versa, characterized in that said movement means (16) are commanded by a central control unit (17) and configured to make said containers (12) advance automatically also along a second advance line (L2) which is autonomous with respect to said first advance line (L1), and in that said apparatus (10) also comprises one or more second washing stations (26) associated with said second advance line (L2), and second pick-up means (71) configured to automatically pick up at least one of said containers (12) from said at least one second advance line (12) and position it in one of said second washing stations (26), and vice versa.

2. Washing apparatus (10) as in claims 1, characterized in that said washing stations (25, 26) comprise at least one of either a cold washing station (27), an ultrasonic washing station (28) and a targeted washing station (29, 30).

3. Washing apparatus (10) as in claim 1, in which at least one advance line (L1, L2) comprises an inlet end (E) and an outlet end (U), characterized in that it also comprises an inlet station (85) disposed in correspondence with said inlet end (E) and configured to allow to position said containers (12) on said movement means (16), and an outlet station (95) disposed in correspondence with said outlet end (U) and configured to allow to pick up said containers (12) from said movement means (16).

4. Washing apparatus (10) as in claim 3, characterized in that said inlet station (85) comprises a robotized anthropomorphic arm (89) configured to pick up a container (12) from a collection zone (86) and position it in the first advance line (L1) or in the second advance line (L2).

5. Washing apparatus (10) as in claim 3, characterized in that said outlet station (95) comprises grouping means (101) configured to position the containers (12) present in said outlet station (95) on at least one mobile support member (99) configured to be inserted in a machine for thermal disinfection (201).

6. Washing apparatus (10) as in claim 5, characterized in that said grouping means (101) comprise two lifting devices (102) each of which is associated with a movement member (20, 21) of said movement means (16) and configured to receive a flow of containers (12) coming from the respective movement member (20, 21).

7. Washing apparatus (10) as in claim 5, characterized in that said grouping means (101) comprise a robotized anthropomorphic arm (105) configured to pick up the containers (12) from said outlet station (95) and position them in a grouping zone (97).

8. Washing apparatus (10) as in claim 3, characterized in that it also comprises a plurality of closing walls (80) containing inside them at least said movement means (16), said washing stations (25, 26) and said pick-up means (70, 71), and having an inlet aperture (81) in correspondence with said inlet end (E) and an outlet aperture (82) in correspondence with said outlet end (U).

9. Washing apparatus (10) as in claim 1, characterized in that with each container (12) there is associated a respective recognition element (14) encoding the information on the instruments (11) contained therein and in that it comprises recognition means (88) configured to recognize the recognition element (14) of each container (12) the instant in which the latter is positioned on the movement means (16).

10. Washing apparatus (10) as in claim 1, characterized in that said pick-up means (70, 71) comprise a plurality of pick-up members (72) each of which is interposed between an advance line (L1, L2) and a washing station (25, 26), wherein each pick-up member (72) comprises a frame (74) on which a mobile gripping member (73) is mounted, commanded by the central control unit (17) and configured to displace a container (12) from an advance line (L1, L2) toward the associated washing station (25, 26) and vice versa.

11. Washing apparatus (10) as in claim 10, characterized in that said gripping member (73) is substantially C-shaped and its internal portion mirrors, negatively, at least one portion of the external perimeter of a container (12).

12. Washing apparatus (10) as in claim 10 [or 11], characterized in that said frame (74) comprises a temporary storage zone (75) on which a container (12) can be disposed so as to compensate for any fluctuations in the flow of containers (12) introduced into the washing apparatus (10).

13. Plant (200) for washing surgical instruments (11) comprising an apparatus (10) as in claim 1, at least one machine (201) for the thermal disinfection of said surgical instruments (11) and additional movement means (202) configured to automatically move said surgical instruments (11) coming out of said apparatus (10) toward said at least one thermal disinfection machine (201).

14. Washing method for washing, in a washing apparatus (10), surgical instruments (11) which are disposed in one or more containers (12), wherein said washing apparatus (10) is provided with movement means (16) which are configured to make said containers (12) advance automatically along at least one first advance line (L1), a plurality of first washing stations (25) associated with said at least one advance line (L1), each of which is configured to perform a washing operation on said surgical instruments (11), and first pick-up means (70) configured to automatically pick up at least one of said containers (12) from said at least one first advance line (L1) and position it in one of said first washing stations (25), and vice versa, wherein said movement means (16) are commanded by a central control unit (17) and configured to make said containers (12) advance automatically also along a second advance line (L2) which is autonomous with respect to said first advance line (L1), and wherein said apparatus (10) also comprises one or more second washing stations (26) associated with said second advance line (L2), and second pick-up means (71) configured to automatically pick up at least one of said containers (12) from said at least one second advance line (L2) and position it in one of said second washing stations (26), and vice versa, wherein the method is characterized in that it comprises:
an introduction step in which said containers (12) are positioned on said first and second advance line (L1, L2);
a feed step in which first containers (12A) are made to advance automatically on said first advance line (L1) and, simultaneously, second containers (12B) are made to advance on said second advance line (L2);
a first pick-up step in which at least one container (12A) is automatically picked up from said first advance line (L1) and positioned in one of said first washing stations (25) and at least one container (12B) is automatically picked up from said second advance line (L2) and positioned in one of said second washing stations (26);
a washing step in which a washing of the surgical instruments (11) contained in said containers (12) is performed;
a second pick-up step in which said containers (12) are automatically picked up from the washing stations (25, 26) in which they are located and positioned in another washing station (25, 26), or in the corresponding advance lines (L1, L2).

15. Method as in claim 14, characterized in that said at least one washing operation is selected from a cold washing operation, an ultrasonic washing operation and a targeted washing operation.
